(19) United States
(12) Patent Application Publication
(10) Pub. No.: US 2023/0076862 A1
(43) Pub. Date: Mar. 9, 2023

(54) ENCAPSULATED DEVICES WITH SEPARATION LAYERS

(71) Applicant: Cook Medical Technologies LLC, Bloomington, IN (US)

(72) Inventor: David C. Majercak, Bloomington, IN (US)

(21) Appl. No.: 17/899,199

(22) Filed: Aug. 30, 2022

Related U.S. Application Data
(60) Provisional application No. 63/240,424, filed on Sep. 3, 2021.

(51) Int. Cl. A61F 2/07

(52) U.S. Cl. CPC A61F 2/07; A61F 2002/072; A61F 2210/0004; A61F 2210/0076

(57) ABSTRACT

Disclosed herein is an encapsulated device that allows for improved flexibility and tailoring to specific needs by a user by selection of features along the lengths and/or circumferences of the encapsulated devices, and that provides enhanced radio-opacity at predetermined locations of the encapsulated device.

### CROSS-REFERENCE TO RELATED APPLICATIONS

This application claims the benefit of U.S. Patent Application Ser. No. 63/240,424, filed Sep. 3, 2021, the entirety of which is incorporated herein by reference for all purposes.

### FIELD

The present disclosure relates generally to medical devices and methods for treating medical conditions, and more specifically, for encapsulated devices including stents and stent-grafts incorporating separation layers for use in body vessels to treat those medical conditions.

### BACKGROUND

Covered stents have come into accepted use for preferred treatment in such applications as bridging stents for aortic stent-grafts and iliac occlusive disease, where the polymeric covering adds to the effective long-term performance of the covered stents.

For the application as a bridging stent for fenestrated and branched stent-grafts, covered stents are used almost exclusively, despite no current covered stent having yet been approved for the bridging indication.

Generally, covered balloon-expandable stents have been preferred over sheathed self-expanding covered stents in fenestrations due to covered balloon-expandable stents having observed advantages, including a lower delivery profile, a higher radial strength, and an improved placement accuracy. However, for branches, primarily due to the observed lack of expanded flexibility, self-expanding covered stents have been preferred.

Thus, there remains a need for further contributions in this area of technology.

### SUMMARY

According to one aspect of the present disclosure, an encapsulated device is provided. The encapsulated device includes at least one stent. The encapsulated device further includes a biocompatible covering encapsulating the at least one stent. The biocompatible covering includes a generally tubular body, a proximal end, a distal end, a central region disposed between the proximal end and the distal end, a lumen extending between the proximal end and the distal end, a first layer, a second layer radially outward from the first layer, and a third layer radially outward from the second layer. The encapsulated device further includes a first separation layer encapsulating at least the central region of the biocompatible covering disposed between the first layer and the second layer of the biocompatible covering. The encapsulated device further includes a second separation layer encapsulating at least the central region of the biocompatible covering disposed between the second layer and the third layer of the biocompatible covering. The biocompatible covering encapsulates the at least one stent internal and/or external to the at least one stent, the at least one stent disposed along a length of the biocompatible covering.

According to another aspect of the present disclosure, an encapsulated device is provided. The encapsulated device includes at least one stent. The encapsulated device further includes a biocompatible covering encapsulating the at least one stent. The biocompatible covering includes a generally tubular body, a proximal end, a distal end, a central region disposed between the proximal end and the distal end, a lumen extending between the proximal end and the distal end, a first layer, a second layer radially outward from the first layer, a third layer radially outward from the second layer, and at least two circumferential zones in an axial direction between the proximal end and the distal end. The encapsulated device further includes a first separation layer encapsulating each of the at least two circumferential zones, the first separation layer disposed between the first layer and the second layer of the biocompatible covering. The encapsulated device further includes a second separation layer encapsulating each of the at least two circumferential zones, the second separation layer disposed between the second layer and the third layer of the biocompatible covering. The biocompatible covering encapsulates the at least one stent internal and/or external to the at least one stent, the at least one stent disposed along a length of the biocompatible covering proximal or distal to each of the at least two circumferential zones.

According to yet another aspect of the present disclosure, an encapsulated device is provided. The encapsulated device includes at least one stent. The encapsulated device further includes a biocompatible covering encapsulating the at least one stent. The biocompatible covering includes a generally tubular body, a proximal end, a distal end, a central region disposed between the proximal end and the distal end, a lumen extending between the proximal end and the distal end, a first layer, a second layer radially outward from the first layer, and a third layer radially outward from the second layer. The encapsulated device further includes a first separation layer encapsulating at least the central region of the biocompatible covering disposed between the first layer and the second layer of the biocompatible covering. The encapsulated device further includes a second separation layer encapsulating at least the central region of the biocompatible covering disposed between the second layer and the third layer of the biocompatible covering. The biocompatible covering encapsulates the at least one stent internal and/or external to the at least one stent, the at least one stent disposed along a length of the biocompatible covering. The first layer is oriented in a circumferential direction about the at least one stent. The second layer is oriented in a second circumferential direction opposite the circumferential direction. The third layer is oriented in the circumferential direction.

Further areas of applicability will become apparent from the description provided herein. It should be understood that the description and specific examples are intended for purposes of illustration only and are not intended to limit the scope of the present disclosure.

### DRAWINGS

In order that the present disclosure may be well understood, there will now be described various forms thereof, given by way of example, reference being made to the accompanying drawings. The components in the figures are not necessarily to scale. Moreover, in the figures, like-referenced numerals designate corresponding parts throughout the different views.

FIG. 1 illustrates a perspective view of the components of an assembly of an example of an encapsulated device according to the principles of the present disclosure;

FIG. 2 illustrates a perspective view of the components of an assembly of another example of an encapsulated device according to the principles of the present disclosure;

FIG. 3 illustrates a perspective view of the components of an assembly of yet another example of an encapsulated device according to the principles of the present disclosure;

FIG. 4 illustrates an end view of yet another example of an encapsulated device assembled according to the principles of the present disclosure;

FIG. 4A illustrates an exploded partial end view of the example of an encapsulated device assembled according to the principles of the present disclosure of FIG. 4;

FIG. 5 illustrates a perspective view of the components of an assembly of yet another example of an encapsulated device according to the principles of the present disclosure;

FIG. 6 illustrates an end view of the encapsulated device of the example of an encapsulated device assembled according to the principles of the present disclosure of FIG. 5;

FIG. 6A illustrates an exploded partial end view of the example of an encapsulated device assembled according to the principles of the present disclosure of FIG. 6; and

FIG. 7 illustrates a perspective view of yet another example of an encapsulated device according to the principles of the present disclosure.

The drawings described herein are for illustration purposes only and are not intended to limit the scope of the present disclosure in any way.

### DETAILED DESCRIPTION

The following description is merely exemplary in nature and is not intended to limit the present disclosure, application, or uses. It should be understood that throughout the drawings, corresponding reference numerals indicate like or corresponding parts and features.

In adding reference denotations to elements of each drawing, although the same elements are displayed on a different drawing, it should be noted that the same elements have the same denotations. In addition, in describing one aspect of the present disclosure, if it is determined that a detailed description of related well-known configurations or functions blurs the gist of one aspect of the present disclosure, it will be omitted.

In the following discussion, the terms “proximal” and “distal” will be used to describe the opposing axial ends of the device, as well as the axial ends of various component features. The term “proximal” is used in its conventional sense to refer to the end of the device (or component) that is closest to the medical professional during use of the assembly. The term “distal” is used in its conventional sense to refer to the end of the device (or component) that is initially inserted into the patient, or that is closest to the patient during use. The term “longitudinal” will be used to refer to an axis that aligns with the proximal-distal axis of the device (or component). The terms “radially” and “radial” will be used to refer to elements, surfaces, or assemblies relative to one another that may extend perpendicularly from a longitudinal axis. The terms “external” and “radially outward,” and “internal” and “radially inward,” will be used to refer to elements, surfaces, or assemblies relative to one another extending perpendicularly from a longitudinal axis, “external” or “radially outward” referring, in context, to elements, surfaces, or assemblies relatively further along a radius from a longitudinal axis than elements, surfaces, or assemblies referred to as “internal” or “radially inward” to such an “external” or “radially outward” element, surface, or assembly. The terms “circumference,” “circumferentially,” and “circumferential” will be used to refer to elements, surfaces, or assemblies relative to one another encircling a longitudinal axis at a radius.

The uses of the terms “a” and “an” and “the” and similar references in the context of describing the present disclosure (especially in the context of the following claims) are to be construed to cover both the singular and the plural, unless otherwise indicated herein or clearly contradicted by context. The use of the term “plurality of” is defined by the Applicant in the broadest sense, superseding any other implied definitions or limitations hereinbefore or hereinafter unless expressly asserted by the Applicant to the contrary, to mean a quantity of more than one. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context.

As used herein the terms “comprise(s),” “include(s),” “having,” “has,” “can,” “may,” “contain(s),” and variants thereof, are intended to be open-ended transitional phrases, terms, or words that do not preclude the possibility of additional acts or structures. The present description also contemplates other embodiments “comprising,” “consisting of,” and “consisting essentially of,” the examples or elements presented herein, whether explicitly set forth or not.

In describing elements of the present disclosure, the terms 1^{st}, 2^{nd}, first, second, A, B, (a), (b), and the like may be used herein. These terms are only used to distinguish one element from another element, but do not limit the corresponding elements irrespective of the nature or order of the corresponding elements.

Unless otherwise defined, all terms used herein, including technical or scientific terms, have the same meanings as those generally understood by those skilled in the art to which the present disclosure pertains. Such terms as those defined in a generally used dictionary are to be interpreted as having meanings equal to the contextual meanings in the relevant field of art.

As used herein, the term “about,” when used in the context of a numerical value or range set forth means a variation of ±15%, or less, of the numerical value. For example, a value differing by ±15%, ±14%, ±10%, or ±5%, among others, would satisfy the definition of “about,” unless more narrowly defined in particular instances.

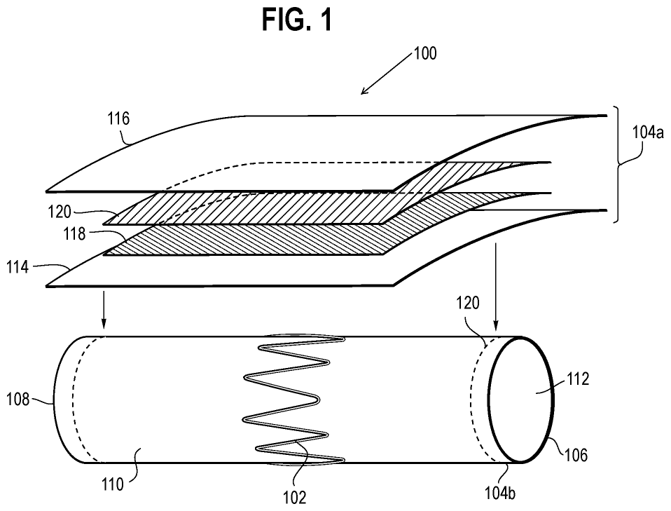

Referring to FIG. 1, a perspective view of the components of an assembly of an example of an encapsulated device 100 according to the principles of the present disclosure is illustrated. Encapsulated device 100 includes at least one stent 102. At least one stent 102 is external to and about the circumference of biocompatible covering 104, which has a generally tubular body. Biocompatible covering 104 extends longitudinally from proximal end 106 to distal end 108 and includes a lumen 112 extending longitudinally from proximal end 106 to distal end 108. Biocompatible covering 104 may include a first portion 104a, the components of which are shown in FIG. 1 being applied to the exterior of at least one stent 102. Biocompatible covering 104 may include a second portion 104b, which is shown assembled to the interior of at least one stent 102 in FIG. 1. Biocompatible covering 104, as illustrated in FIG. 1 by first portion 104a, includes a first layer 114 and a second layer 116, second layer 116 external (i.e., radially outward) relative to first layer 114. Between first layer 114 and second layer 116 are first separation layer 118 and second separation layer 120. FIG. 1 illustrates first layer 114 and second layer 116 of first portion 104a of biocompatible covering 104, with first separation layer 118 and second separation layer 120 together between first layer 114 and second layer 116, being applied external (i.e., radially outward) relative to at least one stent 102. Another first layer 114 and second layer 116 of second portion 104b of biocompatible covering 104, with first separation layer 118 and second separation layer 120 together between first layer 114 and second layer 116, is internal (i.e., radially inward) relative to at least one stent 102. First separation layer 118 and second separation layer 120 encapsulate at least central region 110 of biocompatible covering 104. Though not shown in FIG. 1, first separation layer 118 and second separation layer 120 may extend to proximal end 106 and/or distal end 108 as is preferable or desirable. The lack of adherence and/or bonding of first layers 114 to second layers 116 may contribute to the enhanced flexibility and an added degree of freedom of encapsulated device 100 in that the encapsulated device 100 may move more or less freely with respect to at least one stent 102. Encapsulated device 100 does not suffer the disadvantages of pin-holing and creep resistance known in the art to occur with devices that are not fully encapsulated.

The at least one stent 102 may be made from numerous metals and alloys. In one example, at least one stent 102 includes a shape-memory material such as a nickel-titanium alloy (“nitinol”). Moreover, the structure of at least one stent 102 may be formed in a variety of ways to provide a suitable intraluminal support structure. For example, at least one stent 102 may be made from a woven wire structure, a laser-cut cannula, individual interconnected rings, or another pattern or design.

In another example, as depicted in FIG. 1, at least one stent 102 may be configured in the form of at least one “Z-stents” or Gianturco stents, each of which may include a series of substantially straight segments interconnected by a series of bent segments. The bent segments may include acute bends or apices. The Gianturco stents are arranged in a zigzag configuration in which the straight segments are set at angles relative to each other and are connected by the bent segments. In an example, multiple stents, such as two or more stents, may be discretely spaced-apart in an axial direction from one another between proximal end 106 and distal end 108 of biocompatible covering 104.

First layers 114 and second layers 116 of biocompatible covering 104 may include a polymeric sheet having any suitable porosity. The porosity may be substantially porous or substantially non-porous and may be selected depending on the application. In one example, a porous polymeric sheet may include the polyurethane Thoralon®. In addition to, or in lieu of, a porous polyurethane, examples of biocompatible covering 104 may include, but are not limited to, biocompatible polymeric materials such as non-porous polyurethanes, polytetrafluoroethylene (“PTFE”), expanded PTFE (“ePTFE”), polyethylene tetraphthalate (“PET”), aliphatic polyoxaesters, polylactides, polycaprolactones, and hydrogels. Biocompatible covering 104 may include Dyneema ultra-high molecular weight polyethylene (“UHMwPE”). Biocompatible covering 104 may include a graft material, such as Dacron®, which may optionally be heat treated and/or partially melted.

Where biocompatible covering 104 includes ePTFE, it is advantageous that at least first layer 114 and second layer 116 of biocompatible covering 104 are composed of ultra-thin ePTFE, because there are substantially different mechanical properties between multiple layers of ePTFE and a single layer of ePTFE. At least one layer of biocompatible covering 104 may be internal to at least one stent 102, and at least one layer of biocompatible covering 104 may be external to at least one stent 102, but biocompatible covering 104 may include additional layers as thin as 0.1 microns that are sintered together. Advantageously, one or more separation layers such as first separation layer 118 and second separation layer 120 may be inserted between layers of biocompatible covering 104, which may effectively prevent ePTFE-to-ePTFE bonding.

A separation layer such as first separation layer 118 and second separation layer 120 may include a foil that includes one or more metals. The one or more metals may be radiopaque metals, such that a separation layer is a radiopaque material layer. Examples of radiopaque metals may include a metal or one or more metals selected from the group consisting of gold, platinum, palladium, rhodium, titanium, silver, and tungsten. In case of accidental exposure of a separation layer to blood flow, gold has the advantageous characteristic of being anti-thrombogenic, and silver is known to have anti-bacterial, viral, and inflammatory properties. The foil may be such that where first separation layer 118 and second separation layer 120 are disposed between first layer 114 and second layer 116 of biocompatible covering 104, first layer 114 of biocompatible covering 104 is not adhered to second layer 116. First separation layer 118 and second separation layer 120 may each be up to, or at least, 25 microns thick. Examples of other thickness of first separation layer 118 and second separation layer 120 may include up to, or at least, 50 microns, 75 microns, 100 microns, 125 microns, 150 microns, 175 microns, 200 microns, 225 microns, 250 microns, 500 microns, 750 microns, 1000 microns, 1250 microns, 1500 microns, 1750 microns, 2000 microns, 2250 microns, or 2500 microns. In an example, a single separation layer of 0.15 micron foil would contribute 0.1% of cross-sectional area of a 6F sheath compatible device when wrapped in foil at an 8-millimeter diameter. Encapsulated device 100 may include a third separation layer between biocompatible covering 104 and at least one stent 102.

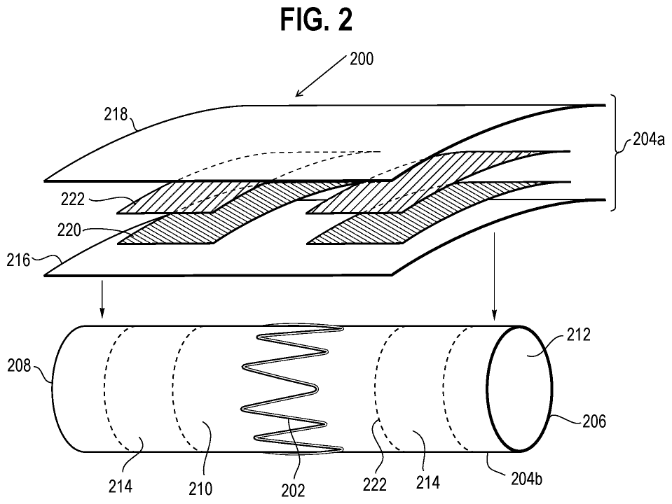

Referring to FIG. 2, a perspective view of the components of an assembly of another example of an encapsulated device 200 according to the principles of the present disclosure is illustrated. Encapsulated device 200 includes at least one stent 202. At least one stent 202 is external to and about the circumference of biocompatible covering 204, which has a generally tubular body. Second portion 204b of biocompatible covering 204 extends from proximal end 206 to distal end 208 and includes lumen 212 extending longitudinally from proximal end 206 to distal end 208. As illustrated in FIG. 2, first portion 204a of biocompatible covering 204 illustrates the assembly of first portion 204a, including a first layer 216 and a second layer 218, second layer 218 external to first layer 216. Between first layer 216 and second layer 218 are first separation layers 220 and second separation layers 222. FIG. 2 illustrates first layer 216 and second layer 218 of biocompatible covering 204, with at least two discrete spaced-apart first separation layers 220 and second separation layers 222, each first separation layer 220 together with a second separation layer 222 between first layer 216 and second layer 218, being applied external relative to at least one stent 202.

Another first layer 216 and second layer 218 of second portion 204b of biocompatible covering 204, with at least two discrete spaced-apart first separation layers 220 and second separation layers 222, each first separation layer 220 together with a second separation layer 222 between first layer 216 and second layer 218, is internal relative to at least one stent 202, illustrated in FIG. 2 as already assembled. The outlines of first separation layers 216 and second separation layers 222 between first layer 216 and second layer 218 are indicated by the at least two circumferential zones 214, which indicate that the first separation layers 220 and second separation layers 222 span the circumference of encapsulated device 200. By including at least two discrete spaced-apart first separation layers 220 and second separation layers 222, each first separation layer 220 together with a second separation layer 222 between first layer 216 and second layer 218, the thicker or multiple layers of foil achieve zones of enhanced radio-opacity. Additionally, the lack of adherence between first layers 216 and second layers 218 of biocompatible covering 204 contributes to the enhanced flexibility and freedom of movement of biocompatible covering 204 relative to at least one stent 202.

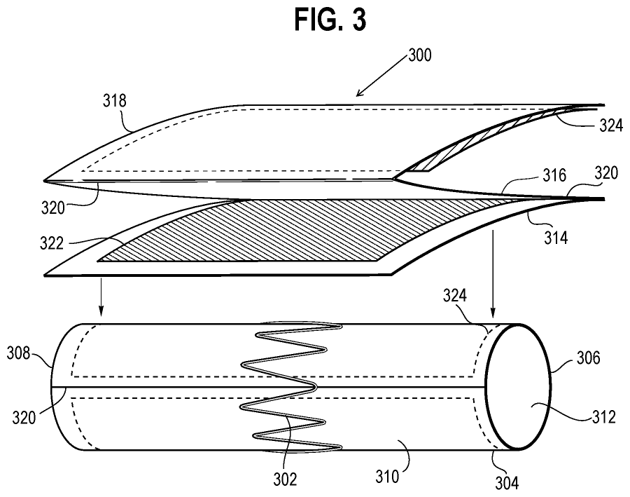

Referring to FIG. 3, a perspective view of the components of an assembly of another example of an encapsulated device 300 according to the principles of the present disclosure is illustrated. Encapsulated device 300 includes at least one stent 302. At least one stent 302 is external to and about the circumference of biocompatible covering 304, which has a generally tubular body. Biocompatible covering 304 extends from proximal end 306 to distal end 308 and includes lumen 312 extending longitudinally from proximal end 306 to distal end 308. Instead of discrete, separate layers as in biocompatible coverings 104 and 204, first layer 314, second layer 316, and third layer 318 of biocompatible covering 304 are formed from a single continuous piece of material(s). First layer 314 is oriented in a circumferential direction about at least one stent 302 until fold 320 in biocompatible covering 304. Fold 320 separates first layer 314 and second layer 316. Second layer 316 is oriented in a second circumferential direction about at least one stent 302, the second circumferential direction opposite the circumferential direction, until fold 320 in biocompatible covering 304. Fold 320 separates second layer 316 and third layer 318. First separation layer 322 is between first layer 314 and second layer 316. Second separation layer 324 is between second layer 316 and third layer 318. First separation layer 322 and second separation layer 324 encapsulate at least central region 310 of biocompatible covering 304. Though not shown in FIG. 3, first separation layer 322 and second separation layer 324 may extend to proximal end 306 and/or distal end 308 as is preferable or desirable. Biocompatible covering 304 with folds 320 separating first layer 314, second layer 316, and third layer 318 such that first layer 314 and third layer 318 are oriented in a circumferential direction and second layer is oriented in a second circumferential direction allow for spots of enhanced visibility under fluoroscopy in predetermined locations to help locate and align encapsulated device 300 as desirable, preferable, or necessary. Additionally, the lack of adherence or bonding between first layers 314 and second layers 316 and between second layers 316 and third layers 318 may contribute to the advantageous enhanced flexibility and freedom of movement of biocompatible covering 304 relative to at least one stent 302.

Another first layer 314, second layer 316, and third layer 318 of biocompatible covering 304, with first separation layer 322 between first layer 314 and second layer 316, and second separation layer 324 between second layer 316 and third layer 318, is internal relative to at least one stent 302. Fold 320 in biocompatible covering 304 internal relative to at least one stent 302 indicates the seam that joins folds 320 between first layer 314 and second layer 316 and second layer 316 and third layer 318. The outline of second separation layer 324 is indicated by central region 310, which indicates that second separation layer 324 nearly spans the circumference of encapsulated device 300.

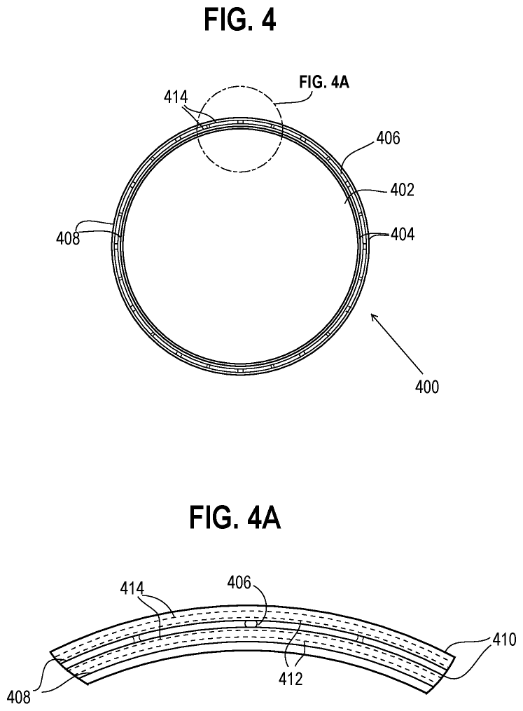

Referring to FIG. 4, an end view of yet another example of an encapsulated device 400 assembled according to the principles of the present disclosure is illustrated. Exploded view FIG. 4A illustrates the relative arrangement of the components of encapsulated device 400 radially relative to one another in more detail. Encapsulated device 400 includes at least one stent 406. Immediately internal (i.e., radially inward) relative to at least one stent 406 is second layer 410 of biocompatible covering 404. Internal to second layer 410 is second separation layer 414. Internal to second separation layer 414 is first separation layer 412. Internal to first separation layer 412 is first layer 408 of biocompatible covering 404. Internal to first layer 408 is lumen 402. Immediately external to at least one stent 406 is first layer 408 of biocompatible covering 404. External to first layer 408 is first separation layer 412. External to first separation layer 412 is second separation layer 414. External to second separation layer 414 is second layer 410 of biocompatible covering 404.

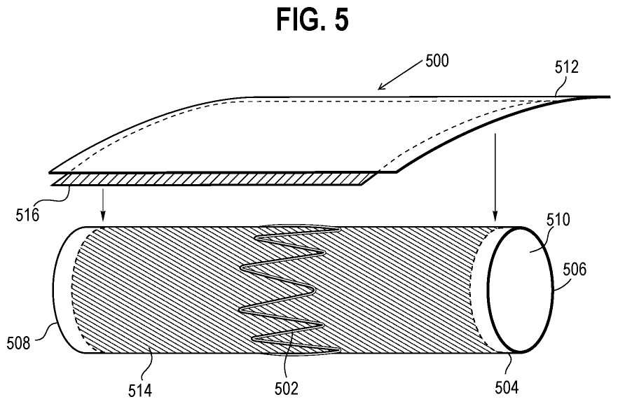

Referring to FIG. 5, a perspective view of the components of an assembly of yet another example of an encapsulated device 500 according to the principles of the present disclosure is illustrated. Encapsulated device 500 includes at least one stent 502. At least one stent 502 is external to and about the circumference of first layer 504 of a biocompatible covering, which has a generally tubular body. First separation layer 514 is internal to at least one stent 502, and between at least one stent 502 and first layer 504 of the biocompatible covering. First layer 504 of biocompatible covering has a generally tubular body and extends from proximal end 506 to distal end 508 and includes lumen 510 extending longitudinally from proximal end 506 to distal end 508. First separation layer 514 encapsulates at least a central region of first layer 504 of the biocompatible covering. Though not shown in FIG. 5, first separation layer 514 may extend to proximal end 506 and/or distal end 508 as is desirable or preferable. External to at least one stent 502 is second separation layer 516. External to second separation layer 516 is second layer 512 of the biocompatible covering. Second separation layer 516 encapsulates at least a central region of first layer 504 of the biocompatible covering in addition to first separation layer 514, and at least one stent 502. Second separation layer 516 encapsulates at least a central region of first layer 504 of the biocompatible covering. Though not shown in FIG. 5, second separation layer 516 may extend to proximal end 506 and/or distal end 508 of first layer 504 or second layer 512 of the biocompatible covering as is desirable or preferable.

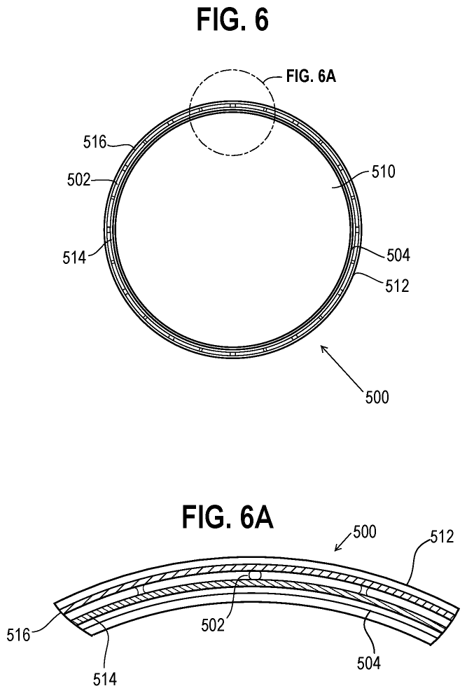

Referring to FIG. 6, an end view of the encapsulated device of the example of an encapsulated device assembled according to the principles of the present disclosure of FIG. 5 is illustrated. Exploded view FIG. 6A illustrates the relative arrangement of the components of encapsulated device 500 radially relative to one another in more detail. Encapsulated device 500 includes at least one stent 502. Immediately internal (i.e., radially inward) relative to at least one stent 502 is first separation layer 514. Internal to first separation layer 514 is first layer 504 of the biocompatible covering. Internal to first layer 504 of the biocompatible covering is lumen 510. Immediately external to at least one stent 502 is second separation layer 516. External to second separation layer 516 is second layer 512 of the biocompatible covering. By including at least one stent 502 between first separation layer 514 and second separation layer 516, adherence of first layer 504 and second layer 512 to at least one stent 502 is prevented.

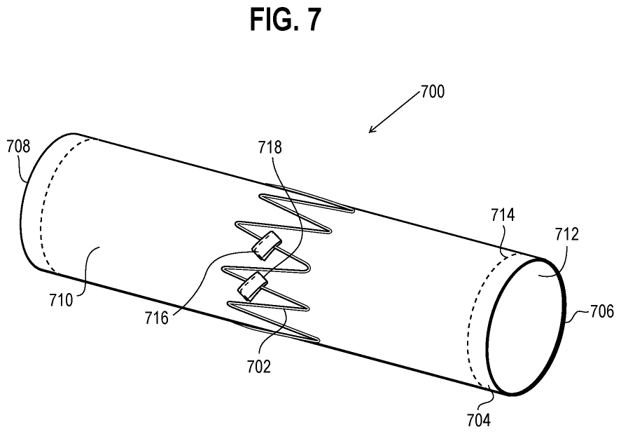

Referring to FIG. 7, a perspective view of yet another example of an encapsulated device 700 according to the principles of the present disclosure is illustrated. Encapsulated device 700 includes at least one stent 702. Wrapped around portions of at least one stent are strips 716 and 718 of a separation layer, which are secured around at least one stent 702 by ends of strip 716 being adhered together and ends of strip 718 being adhered together. Folds in strips 716, 718 allow for spots of enhanced visibility under fluoroscopy in predetermined locations to help locate and align encapsulated device 700 as desirable, preferable, or necessary. At least one stent 702 is external to and about the circumference of biocompatible covering 704, which has a generally tubular body. Biocompatible covering 704 extends from proximal end 706 to distal end 708 and includes lumen 712 extending longitudinally from proximal end 706 to distal end 708. At least one separation layer 714 is internal to biocompatible covering 704, or internal to at least one layer of biocompatible covering 704. At least one separation layer 714 encapsulates at least central region 710 of at least one layer of biocompatible covering 704 and, though not shown in FIG. 7, may extend to proximal end 706 and/or distal end 708 of biocompatible covering 704 as is desirable or preferable.

Encapsulated devices of the present disclosure, including those of the examples illustrated in FIGS. 1-7, advantageously improve flexibility over covered stents known in the art. Prevention of adherence between layers of biocompatible covering according to the examples illustrated in FIGS. 1-7 enhance flexibility and provide for freedom of movement and additional degree of freedom of the biocompatible covering relative to the at least one stent of the examples illustrated in FIGS. 1-7. Further, the encapsulated devices of the present disclosure may be tailored along the lengths and/or circumferences of the encapsulated devices, with the ability to prevent negative aspects as encountered with covered stent devices known in the art. By virtue of being fully encapsulated, the encapsulated devices of the present disclosure do not suffer from the problems of pin-holing and creep resistance known in the art to be experienced by devices that are not fully encapsulated. Further, the encapsulated devices of the present disclosure may provide some enhanced radio-opacity at predetermined locations.

Although the present disclosure has been described with reference to examples and the accompanying drawings, the present disclosure is not limited thereto, but may be variously modified and altered by those skilled in the art, to which the present disclosure pertains without departing from the spirit and scope of the present disclosure.

The subject-matter of the disclosure may also relate, among others, to the following aspects:

A first aspect relates to an encapsulated device, the encapsulated device comprising: at least one stent; a biocompatible covering encapsulating the at least one stent, the biocompatible covering comprising: a generally tubular body, a proximal end, a distal end, a central region disposed between the proximal end and the distal end, a lumen extending between the proximal end and the distal end, a first layer, a second layer radially outward from the first layer, and a third layer radially outward from the second layer; a first separation layer encapsulating at least the central region of the biocompatible covering disposed between the first layer and the second layer of the biocompatible covering; and a second separation layer encapsulating at least the central region of the biocompatible covering disposed between the second layer and the third layer of the biocompatible covering; and wherein the biocompatible covering encapsulates the at least one stent internal and/or external to the at least one stent, the at least one stent disposed along a length of the biocompatible covering.

A second aspect relates to the encapsulated device of aspect 1, wherein the first layer is oriented in a circumferential direction about the at least one stent; wherein the second layer is oriented in a second circumferential direction opposite the circumferential direction; and wherein the third layer is oriented in the circumferential direction.

A third aspect relates to the encapsulated device of any preceding aspect, wherein the first separation layer and the second separation layer extend to at least the proximal end of the biocompatible covering.

A fourth aspect relates to the encapsulated device of any preceding aspect, wherein multiple stents are discretely spaced-apart in an axial direction from one another between the proximal end and the distal end of the biocompatible covering, and wherein the multiple stents are encapsulated by the biocompatible covering.

A fifth aspect relates to an encapsulated device, the encapsulated device comprising: at least one stent; a biocompatible covering encapsulating the at least one stent, the biocompatible covering comprising: a generally tubular body, a proximal end, a distal end, a central region disposed between the proximal end and the distal end, a lumen extending between the proximal end and the distal end, a first layer, a second layer radially outward from the first layer, a third layer radially outward from the second layer, and at least two circumferential zones in an axial direction between the proximal end and the distal end; a first separation layer encapsulating each of the at least two circumferential zones, the first separation layer disposed between the first layer and the second layer of the biocompatible covering; and a second separation layer encapsulating each of the at least two circumferential zones, the second separation layer disposed between the second layer and the third layer of the biocompatible covering; and wherein the biocompatible covering encapsulates the at least one stent internal and/or external to the at least one stent, the at least one stent disposed along a length of the biocompatible covering proximal or distal to each of the at least two circumferential zones.

A sixth aspect relates to the encapsulated device of aspect 6, wherein multiple stents are discretely spaced-apart in the axial direction from one another between the proximal end and the distal end of the biocompatible covering, wherein each of the multiple stents is disposed proximal or distal to each of the at least two circumferential zones, and wherein the multiple stents are encapsulated by the biocompatible covering.

A seventh aspect relates to the encapsulated device of any preceding aspect, wherein the biocompatible covering comprises expanded polytetrafluoroethylene (ePTFE).

An eighth aspect relates to the encapsulated device of any preceding aspect, wherein the first separation layer and the second separation layer each comprise a radiopaque metal selected from the group consisting of gold, platinum, palladium, rhodium, titanium, silver, and tungsten.

A ninth aspect relates to the encapsulated device of any preceding aspect, wherein the first separation layer and the second separation layer each have a thickness of at least 0.001 inch.

A tenth aspect relates to the encapsulated device of any preceding aspect, wherein the device comprises a third separation layer between the biocompatible covering and the at least one stent.

In addition to the features mentioned in each of the independent aspects enumerated above, some examples may show, alone or in combination, the optional features mentioned in the dependent aspects and/or as disclosed in the description above and shown in the figures.

What is claimed is:

1. An encapsulated device, comprising:
at least one stent;
a biocompatible covering encapsulating the at least one stent, the biocompatible covering comprising: a generally tubular body, a proximal end, a distal end, a central region disposed between the proximal end and the distal end, a lumen extending between the proximal end and the distal end, a first layer, a second layer radially outward from the first layer, and a third layer radially outward from the second layer;
a first separation layer encapsulating at least the central region of the biocompatible covering disposed between the first layer and the second layer of the biocompatible covering; and
a second separation layer encapsulating at least the central region of the biocompatible covering disposed between the second layer and the third layer of the biocompatible covering; and
wherein the biocompatible covering encapsulates the at least one stent internal and/or external to the at least one stent, the at least one stent disposed along a length of the biocompatible covering.

2. The encapsulated device of claim 1, wherein the first separation layer and the second separation layer extend to at least the proximal end of the biocompatible covering.

3. The encapsulated device of claim 1, wherein multiple stents are discretely spaced-apart in an axial direction from one another between the proximal end and the distal end of the biocompatible covering, and wherein the multiple stents are encapsulated by the biocompatible covering.

4. The encapsulated device of claim 1, wherein the biocompatible covering comprises expanded polytetrafluoroethylene (ePTFE).

5. The encapsulated device of claim 1, wherein the first separation layer and the second separation layer each comprise a radiopaque metal selected from the group consisting of gold, platinum, palladium, rhodium, titanium, silver, and tungsten.

6. The encapsulated device of claim 1, wherein the first separation layer and the second separation layer each have a thickness of at least 0.001 inch.

7. The encapsulated device of claim 1, wherein the encapsulated device comprises a third separation layer between the biocompatible covering and the at least one stent.

8. An encapsulated device, comprising:
at least one stent;
a biocompatible covering encapsulating the at least one stent, the biocompatible covering comprising: a generally tubular body, a proximal end, a distal end, a central region disposed between the proximal end and the distal end, a lumen extending between the proximal end and the distal end, a first layer, a second layer radially outward from the first layer, a third layer radially outward from the second layer, and at least two circumferential zones in an axial direction between the proximal end and the distal end;
a first separation layer encapsulating each of the at least two circumferential zones, the first separation layer disposed between the first layer and the second layer of the biocompatible covering; and
a second separation layer encapsulating each of the at least two circumferential zones, the second separation layer disposed between the second layer and the third layer of the biocompatible covering; and
wherein the biocompatible covering encapsulates the at least one stent internal and/or external to the at least one stent, the at least one stent disposed along a length of the biocompatible covering proximal or distal to each of the at least two circumferential zones.

9. The encapsulated device of claim 8, wherein multiple stents are discretely spaced-apart in the axial direction from one another between the proximal end and the distal end of the biocompatible covering, wherein each of the multiple stents is disposed proximal or distal to each of the at least two circumferential zones, and wherein the multiple stents are encapsulated by the biocompatible covering.

10. The encapsulated device of claim 8, wherein the biocompatible covering comprises expanded polytetrafluoroethylene (ePTFE).

11. The encapsulated device of claim 8, wherein the first separation layer and the second separation layer each comprise a radiopaque metal selected from the group consisting of gold, platinum, palladium, rhodium, titanium, silver, and tungsten.

12. The encapsulated device of claim 8, wherein the first separation layer and the second separation layer each have a thickness of at least 0.001 inch.

13. The encapsulated device of claim 8, wherein the encapsulated device comprises a third separation layer between the biocompatible covering and the at least one stent.

14. An encapsulated device, comprising:
at least one stent;
a biocompatible covering encapsulating the at least one stent, the biocompatible covering comprising: a generally tubular body, a proximal end, a distal end, a central region disposed between the proximal end and the distal end, a lumen extending between the proximal end and the distal end, a first layer, a second layer radially outward from the first layer, and a third layer radially outward from the second layer;
a first separation layer encapsulating at least the central region of the biocompatible covering disposed between the first layer and the second layer of the biocompatible covering; and
a second separation layer encapsulating at least the central region of the biocompatible covering disposed between the second layer and the third layer of the biocompatible covering; and
wherein the biocompatible covering encapsulates the at least one stent internal and/or external to the at least one stent, the at least one stent disposed along a length of the biocompatible covering;
wherein the first layer is oriented in a circumferential direction about the at least one stent;
wherein the second layer is oriented in a second circumferential direction opposite the circumferential direction; and
wherein the third layer is oriented in the circumferential direction.

15. The encapsulated device of claim 14, wherein the first separation layer and the second separation layer extend to at least the proximal end of the biocompatible covering.

16. The encapsulated device of claim 14, wherein multiple stents are discretely spaced-apart in an axial direction from one another between the proximal end and the distal end of the biocompatible covering, and wherein the multiple stents are encapsulated by the biocompatible covering.

17. The encapsulated device of claim 14, wherein the biocompatible covering comprises expanded polytetrafluoroethylene (ePTFE).

18. The encapsulated device of claim 14, wherein the first separation layer and the second separation layer each comprise a radiopaque metal selected from the group consisting of gold, platinum, palladium, rhodium, titanium, silver, and tungsten.

19. The encapsulated device of claim 14, wherein the first separation layer and the second separation layer each have a thickness of at least 0.001 inch.

20. The encapsulated device of claim 14, wherein the encapsulated device comprises a third separation layer between the biocompatible covering and the at least one stent.
